(19) United States
(12) Patent Application Publication
(10) Pub. No.: US 2005/0196744 A1
(43) Pub. Date: Sep. 8, 2005

(54) Method of screening cocaine antagonists

(76) Inventor: Christopher K. Surratt, Gibsonia, PA (US)

Correspondence Address: Debra Z. Anderson;Eckert Seamans Cherin & Mellott, LLC, 44th Floor, 600 Grant Street, Pittsburgh, PA, 15219, US

(21) Appl. No.: 10/794,375

(22) Filed: Mar. 5, 2004

(51) Int. Cl. C12Q 1/00

(52) U.S. Cl. 435/4

(57) ABSTRACT

The present invention provides a method of screening for antagonists of cocaine and other abused psychostimulants that exert their effects via the dopamine transporter protein, of therapeutic value in treating psychostimulant addiction. More particularly, the invention provides a method of screening for compounds that prevent inhibition of dopamine transport by an abused psychostimulant, using a mutant dopamine transporter protein. The mutant dopamine transporter protein provides the ability to select compounds that not only inhibit cocaine or other drugs from binding to the protein, but also prevent psychostimulant drugs of abuse from interfering with the protein's ability to transport dopamine across the cell membrane.

### FIELD OF THE INVENTION

The present invention relates to a method of screening for antagonists to drugs such as cocaine that affect the dopamine transporter protein, of therapeutic value in treating addiction to cocaine and similar psychostimulants. More particularly, the invention provides a method of screening novel compounds for their potential as therapeutically useful cocaine antagonists using a mutant dopamine transporter protein.

### BACKGROUND INFORMATION

Cocaine is presently the most abused major stimulant in America. It has recently become the drug most frequently involved in emergency department visits. Cocaine initiates its euphoric effects by binding to the dopamine transporter (DAT), blocking uptake of synaptic dopamine. The resultant excess of dopamine in the synapse leads to an increase in postsynaptic receptor binding of the neurotransmitter; this potentiation of the transduced signal eventually leads to addiction to cocaine.

Almost no definitive information exists on the conformation of neurotransmitter transporter proteins, as no crystallographic data are available. No direct contact points have been unequivocally established between a transporter in the Na+/Cl⁻ dependent transporter family and either its cognate neurotransmitter or cocaine, although specific serotonin transporter residues have been identified as contributors to cocaine or mazindol binding sites (Barker E L et al. “High affinity recognition of serotonin transporter antagonists defined by species-scanning mutagenesis: an aromatic residue in transmembrane domain I dictates species-selective recognition of citalopram and mazindol.” J. Biol. Chem. 273:19459-19468 (1998); Chen J G et al. “The third transmembrane domain of the serotonin transporter contains residues associated with substrate and cocaine binding.”J. Biol. Chem. 272:28321-28327 (1997)). Lack of conformational data leads to difficulty in designing drugs that can interact with the transporter proteins.

Studies on the effects of cocaine on the DAT protein indicate that the drug both binds with high affinity to the protein and prevents reuptake of the neurotransmitter dopamine into the presynaptic cell. Candidate cocaine antagonists are typically tested for their ability to block high affinity binding using the wild type DAT protein. However, it has been found that blocking high affinity binding is not necessarily predictive of a drug candidate's ability to block cocaine's action on dopamine reuptake (Wang W et al., “Dissociation of high-affinity cocaine analog binding and dopamine uptake inhibition at the dopamine transporter” Mol. Pharmacol. 64:430-439 (2003); Pristupa Z B et al. “Pharmacological heterogeneity of the cloned and native human dopamine transporter: Dissociation of [³H]WIN 35,428 and [³H]GBR 12,935 binding” Mol. Pharmacol. 45:125-135 (1994)). Thus, prior art methods which focus on screening compounds which block high affinity binding of cocaine or its analogs do not necessarily result in the identification of compounds which are therapeutically useful in blocking the effects of cocaine on dopamine transport.

There remains a need, therefore, for a tool that can aid in the selection of therapeutically useful compounds for the treatment of drug addiction involving the dopamine transport system.

### SUMMARY OF THE INVENTION

The present invention solves the above need, by providing a method of screening compounds that detects the candidate therapeutic's ability to block psychostimulant inhibition of dopamine uptake from the synapse, regardless of the candidate's ability to bind with high affinity to the DAT protein. Previous efforts to discover drugs having this function have been largely unsuccessful, due to the use of the wild type DAT in the screening assays. Compounds having the ability to bind to DAT with high affinity as well as block high affinity binding of cocaine have been identified in assays, but the compounds were found to be lacking in therapeutic value because the compound also effectively blocked dopamine uptake into the presynaptic cell (in essence, served as another version of cocaine). With the discovery of the D79E DAT mutant, it has become apparent that there may be two populations of DAT protein, one population binding cocaine with high affinity, and yet having little transporter function. A second DAT population that binds cocaine with lower affinity appears to be principally responsible for synaptic uptake of dopamine. The D79E mutation is thought to eliminate the population of DAT proteins displaying low transport function and high affinity cocaine binding. In fact, binding affinity values for classic DAT inhibitors such as cocaine, methylphenidate (Ritalin™), mazindol and the cocaine analog WIN 35,428 are very similar or identical to the dopamine uptake inhibition potency values for these drugs for the D79E DAT protein (Wang W et al., Mol. Pharmacol. 64:430-439 (2003)). This result suggests that the drug is binding solely to the DAT population responsible for mediating the drug's psychoactive effects. Use of the mutant DAT protein in screening assays thus allows one to selectively target the physiologically relevant DAT population, and should accurately assess a candidate compound's effectiveness in blocking cocaine or other psychostimulant actions while largely sparing dopamine transport.

It is an object of the present invention, therefore, to provide a method of screening candidate compounds to select a compound useful in the treatment of addiction to cocaine or other abused psychostimulants.

It is an additional object of the present invention to provide a method of screening compounds using cells transfected with a mutant dopamine transporter protein.

It is an additional object of the present invention to provide a method of screening candidate compounds to select a compound that will not inhibit dopamine uptake but will displace cocaine or other abused psychostimulants from the mutant dopamine transporter protein.

These and other objects of the invention will become more readily apparent from the following detailed description and appended claims.

### BRIEF DESCRIPTION OF THE DRAWINGS

The invention is further illustrated by the following drawings in which:

FIG. 1 is a chart comparing [³H]-dopamine uptake ability by COS-7 cells expressing WT or D79 mutant DAT proteins.

FIG. 2 shows assessment of cell surface expression of WT and mutant DAT proteins: (2a) COS-7 cells transiently expressing WT, D79A, D79E or D79N DAT were subjected to a biotinylation agent and processed as described below.

(2b) CHO—K1 cells stably-transfected with WT or D79E DAT were similarly biotinylated, and DAT immunoreactivity of lysates determined.

FIG. 3 is an analysis of WT and D79E DAT dopamine uptake saturation kinetics. CHO cells stably transfected with WT (closed circles) or D79E (open circles) DAT were incubated with increasing concentrations of a fixed ratio of [³H]-dopamine: nonradioactive dopamine. A representative result of 3 separate experiments is shown.

FIG. 4 is an analysis of dopamine or DHBA (a one-carbon shorter version of dopamine) inhibition of [³H]-dopamine uptake (FIG. 4(a)) or [³H]—WIN 35,428 binding (FIG. 4(b)) under identical conditions at CHO cells stably transfected with WT or D79E DAT. The data are representative of at least 3 independent experiments.

FIG. 5 is an analysis of WIN 35,428 (FIGS. 5(a) and 5(b)) or cocaine (FIGS. 5(c) and 5(d)) inhibition of [³H]-dopamine uptake (5(a) and 5(c)) or [³H]—WIN 35,428 binding (5(b) and 5(d)) under identical conditions at CHO cells stably transfected with WT (filled circles) or D79E DAT (open circles). The data are representative of at least 3 independent experiments

FIG. 6 shows cocaine 24-point curves for inhibition of [³H]-dopamine uptake by cells stably transfected with WT (FIG. 6(a), closed circles) or D79E (FIG. 6(b), open circles) DAT. The dashed line indicates a 1-site regression fit. The data are representative of 3 independent experiments.

FIG. 7 is a diagram showing a scenario consistent with the observed dissociation of high affinity WIN 35,428 binding and dopamine uptake inhibition at DAT.

### DETAILED DESCRIPTION OF PREFERRED EMBODIMENTS

The present invention thus provides a method of detecting a compound that is therapeutically useful in the treatment of cocaine and other types of drug addiction involving the dopamine transport system. Such a compound will interfere with cocaine's ability to both a) bind the transporter protein, and b) inhibit dopamine uptake, and yet the compound itself will not substantially inhibit dopamine uptake (e.g. act as a cocaine substitute). It has been found that therapeutically useful compounds in the treatment of other drug addictions such as heroin, for example, do not provide 100% reduction in the pleasurable symptoms provided by the drug. This is therapeutically useful, because a person physically dependent on an abused drug often needs incentive to maintain a treatment regimen. Such therapeutics allows gradual withdrawal from the abused drug without eliciting the “withdrawal syndrome” (e.g., profound nausea, fever, chills). As used herein, the term “interfere with”, when used in connection with the candidate compound's ability to displace cocaine or other drug from the transporter protein, will refer to candidate compounds that provide more than a 60-95%, preferably more than a 70-95% reduction in the action of cocaine or other DAT-binding psychostimulants, while independently acting on the dopamine transporter protein to allow dopamine uptake even in the presence of the drug. Thus, for example, a therapeutically useful candidate may be one that provides about a 90% reduction in the action of the drug. Similarly, the term “not substantially inhibit”, as used herein in connection with the action of the candidate compound on dopamine uptake, will refer to the fact that a desirable candidate may allow some low level of inhibition of dopamine uptake. Preferably, the level of inhibition is less than about 30-40%; most preferably, the level of inhibition is less than about 5-20%.

The method comprises providing one or more cells transfected with a mutant dopamine transporter protein and expressing the protein, contacting the cells with a first compound, the candidate antagonist compound of interest, potentially able to interfere with the effects of cocaine or other abused drug on dopamine transport, and a second compound representing the drug for which treatment is desired (e.g., cocaine). The ability of the first compound to displace cocaine or other abused psychostimulants, as determined by binding affinity, is measured. As used herein, binding affinity refers to the concentration of candidate drug necessary to displace approximately 50% of cocaine, its analogs, or other abused psychostimulants from the DAT protein.

Dopamine uptake inhibition potency is also measured in the presence of both the first and second compounds, as well as in the presence of each compound separately. A therapeutically useful compound will minimally inhibit dopamine uptake, but will displace cocaine or other abused psychostimulants from the mutant dopamine transporter protein. One skilled in the art can identify suitable compounds on the basis of the binding affinity and dopamine uptake inhibition potency measurements. Measurements of cocaine (or analog or other psychostimulant) binding affinity and dopamine uptake inhibition potency in the absence of the candidate compound are conducted for comparison purposes to determine the effectiveness of the candidate compound.

In one embodiment, binding affinity and dopamine uptake inhibition potency measurements are both carried out. In another embodiment, either endpoint can be measured separately.

Preferably, the mutant dopamine transporter protein is D79E DAT. Other mutant dopamine transporter proteins can be used, and are contemplated as embraced by the present invention, so long as the mutant provides the ability to distinguish between high affinity binding of cocaine and the effects of cocaine on the dopamine transporter function of the protein. One skilled in the art can identify suitable mutant transporter proteins by the assays used to measure cocaine binding affinity and dopamine uptake.

Methods of transfection of the target cells with the protein of interest are well known in the art. Any cells that lack an endogenous monoamine transporter protein and can be transfected with the mutant dopamine transporter can be used, so long as there is sufficient expression of the protein. Preferably, the cells are mammalian cells; most preferably they are CHO cells. Other suitable cell types include, but are not limited to, COS, HEK293, NIH3T3, LLC—PK and PC12 cells.

DAT cell surface expression levels can be determined by cell surface protein labeling methods (see experimental methods below) and subsequent quantitation via densitometric analysis of exposed X-ray film or Western blots treated with chemiluminescent or other signals that indicate the presence of anti-DAT antibody. Any level of expression is suitable, so long as it results in detectable levels of binding and uptake.

Preferably, the drug is selected from cocaine, cocaine analogs such as WIN 35,428, mazindol, methylphenidate, trihexyphenidyl, BTCP, GBR-12909, GBR-12783 or other available radioligands. Due to the low stability of cocaine in vitro, a commonly used and more stable analog of cocaine, WIN 35,428 can be used for screening purposes. Other suitable cocaine analogs include mazindol, methyl-phenidate, trihexyphenidyl, BTCP, GBR-12909, GBR-12783 or other available radioligands. Typically, about 1 picomole to 1 millimole of the candidate compound, and about 1 nanomole to 1 picomole of drug (abused psychostimulant) compound are used in the assays. One skilled in the art can determine the appropriate amount of the compounds for use in contacting the cells.

In a preferred embodiment, two sets of assays are conducted in parallel, using both the mutant DAT and the wild type DAT. The results of the assays are compared to ensure that the values for binding and uptake inhibition are similar for the candidate compound using the mutant DAT and that the candidate is effective at the wild type DAT.

### EXAMPLES

The following examples are intended to illustrate the invention and should not be construed as limiting the invention in any way.

### Materials and Methods

Materials. [³H]—WIN 35,428 (˜85 Ci/mmol) and [³H]-dopamine (˜29 Ci/mmol) were obtained from NEN Life Science Products (Boston, Mass.). Nonradioactive WIN 35,428, cocaine, methylphenidate and amphetamine were obtained from Research Triangle Institute (Research Triangle Park, N.C.) via the National Institute on Drug Abuse Division of Basic Research. Nonradioactive dopamine and ascorbic acid were obtained from Sigma Chemical Co. (St. Louis, Mo.), mazindol, norepinephrine, and the tyramines were obtained from RBI/Sigma (Natick, Mass.), and dihydroxybenzylamine was obtained from Aldrich (Milwaukee, Wis.). Scintillation counting materials were from Fisher Scientific (Pittsburgh, Pa.), and GF/B paper was from Brandel (Gaithersburg, Md.). Anti-DAT antibody (rat) was obtained from Chemicon (Temecula, Calif.), biotinylation reagents were obtained from Pierce Chemical Co. (Rockford, Ill.), and all other Western blotting reagents were from Bio-Rad (Richmond, Calif.). COS-7 and CHO—K1 cell lines were obtained from American Type Culture Collection (Manassas, Va.).

Mutagenesis and cell transfections. All site-directed mutagenesis was conducted as described (Spivak C E, Beglan C L, Seidleck B K, Hirshbein L D, Blaschak C J, Uhl G R and Surratt C K (1997) Naloxone activation of μ opioid receptors mutated at a histidine residue lining the opioid binding cavity. Mol. Pharmacol. 52:983-992). COS-7 cells were cultured in “DMEM” (DMEM plus 10% fetal bovine serum, 10% penicillin/streptomycin and 20 mM L-glutamine) at 37° C. in 5% CO₂ in 750 cm² flasks. Cells were distributed in 24-well plates such that the monolayer would be 95% confluent when the transfection commenced. For an individual well of the 24-well plate, 1 μg plasmid and 1.5 ml of “LIPO 2000” lipofectamine suspension were preincubated 20 min before adding to the well in Optimem medium. After 5 hr, an equal volume of “DMEM” was added to the well, and cells were used for experiments approximately 24 hr after initiating the transfection. Stably-transfected DAT-CHO cell lines were prepared by lipofectamine-mediated transfection and selection of stable transfectants in the presence of 500 μg/ml G-418 in F-12 HAM medium; cell lines were maintained in the same medium containing 100 μg/ml G-418.

Cell surface labeling of expressed DAT proteins. Stably-transfected CHO cells or transiently-transfected COS-7 cells were grown to confluence on 6 well (35 mm/well) polylysine-free plates. After washing the cell monolayer 3×10 min with ice cold phosphate-buffered saline supplemented with 0.1 mM CaCl₂ and 1 mM MgCl₂ (PBS/Ca/Mg buffer) (Ramamoorthy S et al. “Biosynthesis, N-glycosylation, and surface trafficking of biogenic amine transporter proteins.” Meth. Enzymol. 296:347-370, 1998), 1 ml of a 1.5 mg/ml solution of freshly-prepared sulfosuccinimidyl-2-(biotinamido)ethyl-1,3-dithiopropionate in PBS/Ca/Mg buffer was added for 25 min at 4° C. Free biotinylation reagent was quenched by washing thrice with cold PBS/Ca/Mg containing 100 mM glycine. After two more washes with PBS/Ca/Mg alone, cells were lysed with 0.2 ml of 0.1% SDS, 1% Triton X-100, 150 mM NaCl and 1 mM EDTA in 10 mM Tris-HCl, pH 7.4 for 30 min on ice. Lysates were cleared by centrifuging at 14,000×g for 10 min at 4° C. The upper 150 μl of supernatant was transferred to a new tube and combined with 50 μl of NeutrAvidin resin (50% slurry), incubating overnight at 4° C. and mixing with end-over-end rotation. The resin was concentrated to a pellet with 5000×g centrifugation for 4 min at 4° C. The pellet was washed first 3 times by resuspending the resin in the lysis buffer, and then 2 times more in 0.1% Triton X-100, 500 mM NaCl and 5 mM EDTA in 50 mM Tris-HCl, pH 7.5; each wash was centrifuged as before with all steps at 4° C. After a final wash with 50 mM Tris-HCl, pH 7.5, the supernatant was completely removed, and biotinylated proteins were released from the NeutrAvidin resin by addition of standard SDS gel loading buffer containing 2-mercaptoethanol. Biotinylated DAT was separated from other biotinylated proteins by SDS—PAGE, and identified with Western blotting (standard conditions, using a rat anti-DAT primary antibody diluted 1:4000). To test that biotinylation was specific for cell surface proteins, the same blot was stripped of antibodies with 100 mM 2-mercaptoethanol and 2% SDS in 62.5 mM Tris-HCl, pH 6.7, for 1 min at 40° C., and subsequently reprobed with a monoclonal antibody against actin, an intracellular marker protein.

[³H]-Dopamine uptake. Assays were conducted with cell monolayers in 6-well plates. The monolayer was washed 2×2 ml with “KRH buffer” (25 mM HEPES, pH 7.3, 125 mM NaCl, 4.8 mM KCl, 1.3 mM CaCl₂, 1.2 mM Mg₂SO₄, 1.2 mM KH₂PO₄, 5.6 mM glucose), and uptake was typically initiated by addition of 1 ml of 10 nM [³H]-dopamine and 50 μM ascorbic acid (AA) in KRH to duplicate or triplicate cell monolayers. A time course was conducted for each DAT mutant to determine when uptake velocity was in the linear phase, typically occurring at 5-10 minutes at 22° C. Uptake was quenched by washing the monolayer with 2×2 ml of KRH+AA. Cell monolayers were solubilized in 0.5 ml of 1% SDS, and transferred to scintillation vials for determination of incorporated tritium. DAT mutants demonstrating the ability to transport dopamine in this assay were studied in uptake “saturation” experiments in which final concentrations of 0.1-16 μM dopamine were employed, and [³H]-dopamine was diluted with nonradioactive dopamine to obtain a specific activity of 0.1 Ci/mmol. Nonspecific uptake was assessed by inclusion of 30 μM cocaine. Uptake inhibition experiments used 2-20 μM [³H]-dopamine plus nonradioactive DAT uptake blockers at the following concentration ranges: Cocaine, 0.01-100 μM; WIN 35,428, 3-10,000 nM; mazindol, 3-10,000 nM; methylphenidate, 3-10,000 nM; DHBA, 0.3-100 mM. All nonsubstrate inhibitors were preincubated 10 min with the cell monolayer before adding [³H]-dopamine. Kₘ and Vₘₐₓ values for transport and Kᵢ values for uptake inhibition were determined with GraphPad Prism 2.0 software.

Ligand binding assays. [³H]—WIN 35,428, structurally similar to cocaine but much more stable in vitro, was the radioligand employed for all experiments. With the exception of the membrane binding experiment referred to in Results, all binding assays were conducted exactly as described above for the dopamine uptake assay except that [³H]-dopamine was replaced with 1 nM [³H]—WIN 35,428, and radioligand and nonradioactive competitor were incubated with cells for 15 min (the same incubation period allowed for an uptake blocker in the uptake assay). Nonradioactive competitor concentrations were as indicated above for uptake inhibition. Nonspecific binding was assessed by addition of 10 μM mazindol except when mazindol was the drug tested, in which case 50 μM cocaine was used to assess nonspecific binding. For the membrane binding experiment, membranes of CHO cells stably transfected with WT or D79E DAT cDNAs were prepared by suspending 2.5×10⁸ cells in 40 ml of 10 mM HEPES, pH 8.0, 50 mM MgCl₂ buffer, Polytron-homogenizing the suspension, and centrifuging the homogenate 20 min at 48,000×g (all steps at 4° C.). After protein quantitation, membranes were resuspended in KRH buffer and combined with 2-20 nM [³H]—WIN 35,428 and increasing concentrations of nonradioactive WIN 35,428 for 60 min at 22° C. Membranes were aspirated with a Brandel harvester onto GF/B glass-fiber filter paper, washed with 3×5 ml cold binding buffer, and the filters measured for radioactivity via scintillation counting. For all binding assays, data were analyzed with the GraphPad Prism 2.0 software to obtain K_{d}, Kᵢ and Bₘₐₓ values.

Site-directed mutagenesis of the rat DAT D79 residue was employed to define features of the TM 1 aspartic acid side chain important for both dopamine uptake and recognition of drugs that inhibit uptake. Substitution with glutamic acid (D79E), asparagine (D79N) or alanine (D79A) addressed the importance of a negative charge, hydrogen-bonding potential, and side chain length at this position.

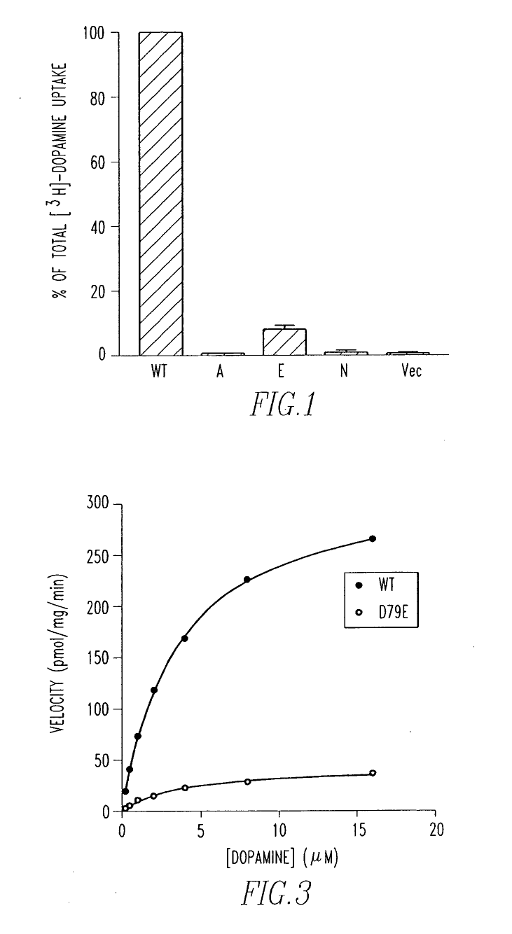

Transiently transfected COS-7 cells were screened for the ability to take up 10 nM [³H]-dopamine (FIG. 1). The extent of total [³H]-dopamine uptake as a percentage of WT DAT was assessed for cells transiently transfected with plasmids encoding D79A (“A”), D79E (“E”) or D79N (“N”) DAT, or the plasmid vector lacking DAT sequence (“Vec”). The data represent an average of 3 separate experiments.

No specific uptake was detected for cells bearing the D79A or D79N DAT mutants, as levels of [³H]-dopamine incorporation did not differ appreciably from that for cells transfected with the pIRES vector alone. D79E-transfected cells, in contrast, clearly exhibited [³H]-dopamine uptake, albeit at a level well below cells bearing “wild type” (wild type+N-terminal epitope tag; WT) DAT. The N-terminally epitope-tagged DAT was pharmacologically indistinguishable from wild-type DAT (data not shown). The introduced epitope was not employed in these experiments, because antisera directed against an authentic DAT N-terminal epitope proved superior for the biotinylation studies.

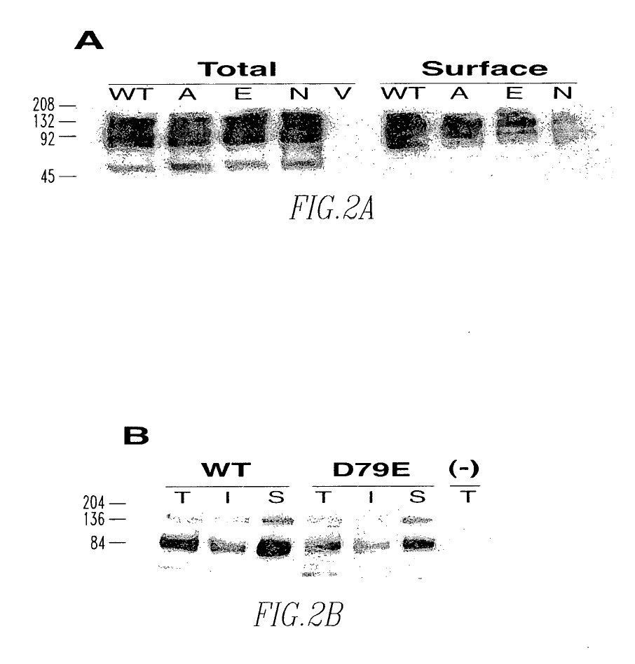

Referring now to FIG. 2(a), total cell lysate (“T”), the intracellular fraction of the lysate (“I”), and the biotinylated and affinity-purified cell surface fraction (“S”) were applied to the polyacrylamide gel. DAT protein was visualized following Western transfer with the aid of a DAT-selective antibody directed against an endogenous amino acid sequence of the N-terminus. No DAT immunoreactivity was observed for the total cell lysate of cells transfected with the plasmid vector alone (“V”). A triplet of immnunoreactive “surface” bands was observed, presumably corresponding to mature (fully glycosylated) multimer (>200 kD), mature monomer (˜120 kD) and an immature monomer (˜95 kD). “Total” lanes additionally contain a band corresponding to a ˜70 kD polypeptide, presumably the unglycosylated monomer. Quantitation of bands was achieved using scanning densitometry, using shorter exposures than those shown in the figure to ensure that the scan was in the linear range of the film. In FIG. 2a, the “total” and “surface” band densities (expressed as a % of WT) were 94% and 76% for D79A DAT, 107% and 61% for D79E DAT, and 114% and 16% for D79N DAT, respectively. The inability of D79A DAT to transport dopamine could not be accounted for by poor surface expression (FIG. 2a), suggesting that a negative charge, hydrogen bonding potential, a longer side chain, or a combination of these is required at Position 79 of DAT for dopamine uptake.

The total D79N DAT expression level was equal to or better than the other DAT proteins, but its surface expression was relatively low—sufficiently lower than that of D79E DAT to account for the functional difference between these mutants. It was therefore not possible to infer that a negatively charged side chain at Position 79 is critical for dopamine transport (the failure of D79A DAT may be due to the shorter side chain or the lack of H-bonding potential). Extracellular biotinylation of the exclusively intracellular β-actin protein was not detected with an anti-actin monoclonal antibody at intact cells, and was only observed for cell lysates (data not shown), meaning that the immunoreactivity of DAT proteins could not be explained by intracellular biotinylation due to cell permeation with biotin.

To more thoroughly characterize D79E DAT relative to WT DAT, stably-transfected CHO—K1 cells were prepared. Biotinylation experiments again established that WT and D79E DAT proteins were present at the cell surface, but total, intracellular, and cell surface expression of D79E DAT were approximately half that of WT DAT (FIG. 2b). Total cell lysates of untransfected CHO cells (“Naïve”) displayed no DAT immunoreactivity. The major band indicated an apparent molecular weight of 94 kD, presumably the mature monomer. A much weaker band of ˜200 kD is evident, consistent with a DAT dimer. The apparent molecular weights of both COS— and CHO-expressed mature monomers are in agreement with published values (Patel A P, Neurotransmitter Transporters: Structure, Function, and Regulation, 1^{st} Ed., Humana Press Inc., Totowa, N.J., 1997). In the figure, the “total”, “intracellular” and “surface” band densities (expressed as a % of WT) for D79E DAT were 51%, 36% and 48%, respectively. A representative result of 3-6 experiments is shown.

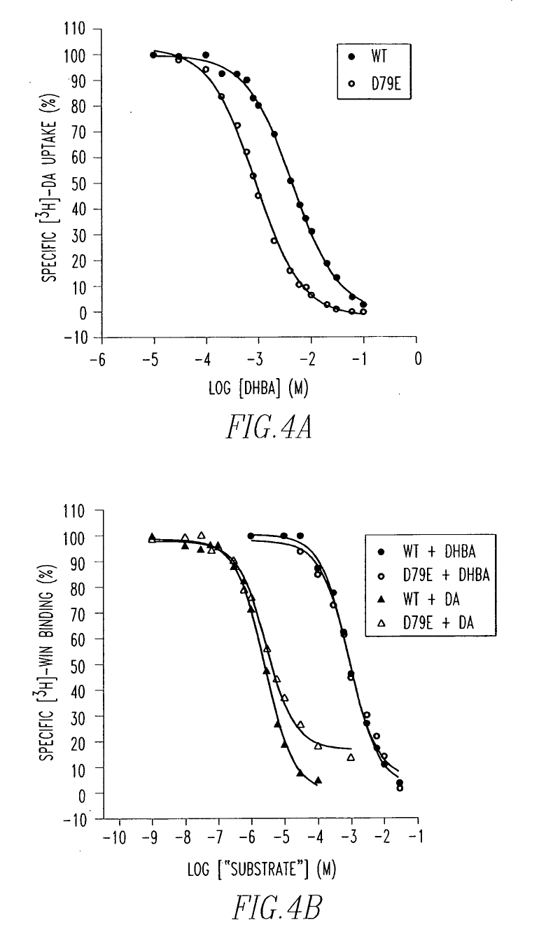

The kinetics of [³H]-dopamine uptake were determined for each stable cell line (FIG. 3). The WT DAT turnover rate in CHO cells was in agreement with that reported for hDAT in oocytes (Sonders M S et al. “Multiple ionic conductances of the human dopamine transporter: The actions of dopamine and psychostimulants”. J. Neurosci. 17:960-974, 1997) and rDAT in LLC—PK cells (Gu H et al. “Stable expression of biogenic amine transporters reveals differences in inhibitor sensitivity, kinetics, and ion dependence.” J. Biol. Chem. 269:7124-7130, 1994), and 7-fold higher than that for D79E DAT CHO cells (Table 1).

Half of the Vₘₐₓ decrease at D79E DAT cells could be attributed to fewer transporter proteins at the cell surface, the latter evidenced by the biotinylation data (FIG. 2b) and supported by the Bₘₐₓ values for [³H]—WIN 35,428 binding (Table 1). Thus, the D79E mutation rendered an approximately 4-fold decrease in DAT dopamine turnover rate (Table 1). A decrease in transport capacity was also observed for the analogous TM 1 SERT mutant D98E (Barker E L et al. “Transmembrane domain I contributes to the permeation pathway for serotonin and ions in the serotonin transporter.” J. Neurosci. 19:4705-4717, 1999).

TABLE 1
Kinetics of dopamine uptake by CHO cells
stably expressing WT or D79E DAT

| | WT DAT | D79E DAT |
|---|---|---|
| Vₘₐₓ (pmol/mg/min) | 348 ± 22 | 48 ± 4^{a} |
| Bₘₐₓ (pmol/mg) | 10.2 ± 0.4 | 6.1 ± 0.6 |
| Vₘₐₓ/Bₘₐₓ | 34.1/min | 7.9/min |

Vₘₐₓ values in Table 1 were derived from experiments incubating the stably-transfected cells with dopamine at 0.1 Ci/mmol for 5 min at 22° C. Bₘₐₓ values were derived from [³H]—WIN 35,428 saturation binding. Mean±standard error for 3 independent experiments. ^{a}p<0.05 versus WT DAT (Student's T test).

TABLE 2
Effect of DAT substrates on CHO cells
stably expressing WT or D79E DAT

Kₘ or Kᵢ (μM)

| | WT DAT | D79E DAT |
|---|---|---|
| [³H]-DA uptake | | |
| Dopamine | 1.7 ± 0.3 | 2.3 ± 0.3 |
| DHBA | 5760 ± 540^{c} | 782 ± 22^{a,c} |
| Norepinephrine | 5.4 ± 0.5^{c} | 5.2 ± 0.3^{c} |
| m-Tyramine | 1.4 ± 0.3 | 1.2 ± 0.3^{c} |
| p-Tyramine | 1.7 ± 0.1 | 3.3 ± 0.4^{a} |
| (+)-Amphetamine | 0.5 ± 0.1 | 2.4 ± 0.3^{a} |
| (−)-Amphetamine | 2.8 ± 0.5 | 4.2 ± 0.5^{c} |
| [³H]-WIN inhibition | | |
| Dopamine | 2.1 ± 0.2 | 2.5 ± 0.4 |
| DHBA | 801 ± 22^{b,c} | 791 ± 27^{c} |
| Norepinephrine | 2.3 ± 0.5^{b} | 5.5 ± 0.7^{a,c} |
| m-Tyramine | 1.0 ± 0.2^{c} | 6.6 ± 1.2^{a,b,}^{c} |
| p-Tyramine | 0.8 ± 0.1^{b,c} | 4.3 ± 0.5^{a} |
| (+)-Amphetamine | 0.4 ± 0.1^{c} | 3.7 ± 0.8^{a} |
| (−)-Amphetamine | 2.0 ± 0.2 | 8.1 ± 0.5^{a,b,c} |

Kₘ and Kᵢ values in Table 2 were derived from experiments incubating the stably-transfected cells with nonradioactive substrates in the presence of [³H]-dopamine or [³H]—WIN 35,428 at 22° C. in KRH buffer. Mean±standard error for 3-6 independent experiments. ^{a}p<0.05 vs. WT DAT for that assay. ^{b}p<0.05 for WIN inhibition vs. dopamine uptake at the same DAT construct. ^{c}p<0.05 vs. dopamine for the given assay and DAT construct. (All 3 statistical analyses via Student's T test.)

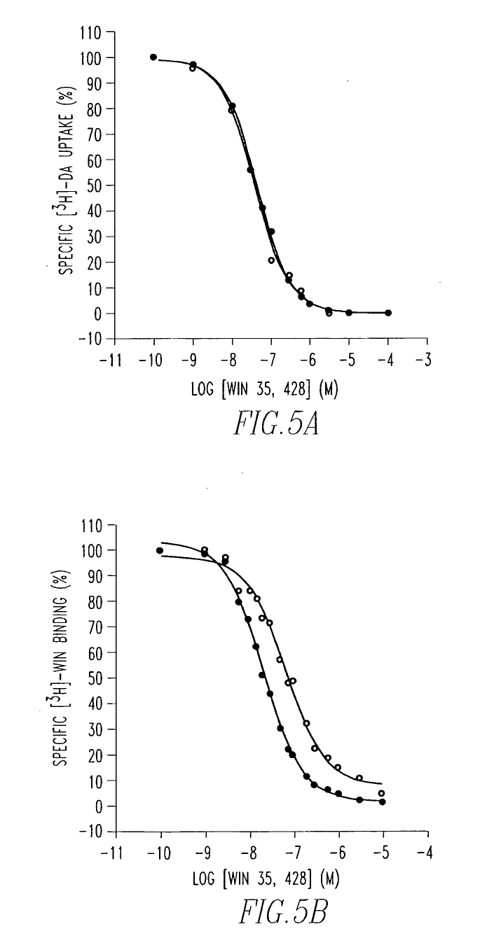

On the other hand, this mutation had little effect on the Kₘ value for dopamine uptake (FIG. 3 and Table 2). Because this value cannot be equated with an affinity constant, dopamine affinity was assessed by its ability to displace [³H]—WIN 35,428 binding at WT and D79E DAT. (Binding affinity Kᵢ values for all other substrates and inhibitors in the study were also assessed in this way, and this Kᵢ value will hereafter be referred to as the “binding affinity” of the ligand for WT or D79E DAT.) By this measure, dopamine displayed an almost identical binding affinity for WT and D79E DAT proteins (FIG. 4 and Table 2). Because intact naïve CHO cells possess a low affinity site (K_{d}>1 μM) for [³H]—WIN 35,428, it was not possible to completely displace radioligand specific binding unless very high concentrations of inhibitor were employed. This result may be inconsistent with the idea that the D79 residue is the governing element of the DAT dopamine binding pocket because the added steric bulk of the glutamate side chain would leave less room for dopamine (assuming that dopamine does not adopt an energetically-unfavorable conformation to accommodate the mutation). To further address this steric issue, dihydroxy-benzylamine (DHBA) was employed as a potential DAT substrate. This compound is identical to dopamine except for the absence of one of two methylene groups between the amino and dihydroxyphenyl groups. If the governing DAT-dopamine point of contact were at D79, and the role of the D79 residue were simply to form an interaction with the amino group of dopamine, the extra methylene group in the glutamic acid side chain would exactly compensate for the shorter DHBA ligand. Binding and uptake of DHBA by D79E DAT should thus be as efficient as dopamine binding and uptake at WT DAT, and superior to that of dopamine at D79E DAT. This was not the case, as DHBA binding affinities were identical at WT and D79E DAT, and DHBA remained a quite inferior inhibitor of [³H]-dopamine uptake (FIG. 4 and Table 2). The potency of DHBA in inhibiting [³H]-dopamine uptake was 7-fold higher at D79E DAT compared to WT DAT. Still, this Kᵢ value for DHBA at D79E DAT was 460-fold higher than the dopamine Kₘ value at WT DAT and 377-fold higher than the Kᵢ value for dopamine binding at WT DAT. The DHBA Kᵢ value for [³H]-dopamine uptake inhibition at D79E DAT agrees well with that for inhibition of [³H]—WIN 35,428 binding; curiously, the [³H]-dopamine uptake inhibition potency of DHBA at WT DAT was 7-fold less than its apparent affinity for inhibiting [³H]—WIN 35,428 binding. Ligand binding assays were conducted under conditions that matched those for assaying uptake inhibition.

Other dopamine analogs assessed the effect of adding substituents to the methylene groups or of altering the catechol hydroxyl groups (Table 2). Norepinephrine (addition of a β-OH group to dopamine) was equally effective at WT and D79E DAT in inhibiting dopamine uptake, but was 2-fold less effective at displacing [³H]—WIN 35,428 at D79E DAT than at WT DAT. The dopamine uptake inhibition potencies of m- or p-tyramine (each lacking one of the catechol OH groups) were unchanged or 2-fold lower as a result of the D79E mutation, respectively, yet the mutation decreased binding affinities for both tyramines by over 5-fold. Of the DAT substrates tested, the D79E mutation most dramatically affected the dopamine uptake inhibition potency (5-fold loss) and binding affinity (9-fold loss) of the more psychoactive isomer of amphetamine (lacking both catechol OH groups and adding an α-methyl group), S(+)-amphetamine. For R(−)-amphetamine, however, dopamine uptake inhibition potency was not significantly affected by the mutation.

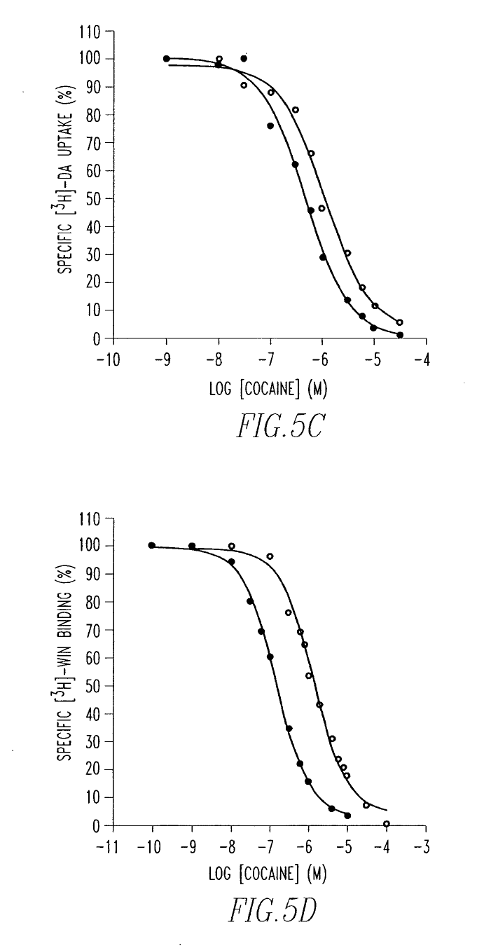

The dopamine uptake inhibition potencies of the classical uptake inhibitors cocaine, WIN 35,428, mazindol and methylphenidate were compared at WT and D79E DAT cells. Of the four inhibitors, only the dopamine uptake inhibition potency of cocaine was altered (2-fold decrease) in a statistically-significant fashion by the mutation (FIG. 5 and Table 3). Binding affinities for each inhibitor were also determined, using intact cell monolayers and under conditions essentially identical to those for dopamine uptake assays. All inhibitors were shown to reach equilibrium with WT or D79E DAT within 15 minutes, as Kᵢ values did not differ from those obtained after 60 or 120 minute binding assays (data not shown). For all four inhibitors, the potency of inhibition of (³H]—WIN 35,428 binding matched the potency for inhibition of [³H]-dopamine uptake at D79E DAT, as would be expected if occupancy of the [³H]—WIN 35,428 binding site prevents substrate translocation. In contrast to the D79E mutant, inhibitory potencies derived from uptake assays and binding assays did not match for intact cells stably expressing WT DAT: WIN 35,428, mazindol, methylphenidate and cocaine were 3- to 4-fold more potent as inhibitors of [³H]—WIN 35,428 binding than as inhibitors of [³H]-dopamine uptake (FIG. 5 and Table 3). These results suggest the existence of a conformational state or binding site in WT DAT with high affinity for WIN 35,428 that is not detectable in D79E DAT.

TABLE 3
Effect of classical DAT inhibitors on CHO
cells stably expressing WT or D79E DAT.

K_{d} or Kᵢ (nM)

| | WT DAT | D79E DAT |
|---|---|---|
| [³H]-DA uptake | | |
| WIN 35,428 | 74 ± 17 | 71 ± 12 |
| Cocaine | 555 ± 29 | 1126 ± 32^{a} |
| Mazindol | 31 ± 3 | 47 ± 4 |
| Methylphenidate | 152 ± 20 | 161 ± 9 |
| [³H]-WIN inhibition | | |
| WIN 35,428 | 20 ± 1^{b} | 68 ± 4^{a} |
| Cocaine | 128 ± 5^{b} | 1066 ± 84^{a} |
| Mazindol | 13 ± 1^{b} | 41 ± 3^{a} |
| Methylphenidate | 57 ± 3^{b} | 167 ± 2^{a} |

K_{d} (WIN 35,428 only) and Kᵢ values in Table 3 were derived from experiments incubating the stably-transfected cells with nonradioactive substrates in the presence of [³H]-dopamine or [³H]—WIN 35,428 at 22° C in KRH buffer. Values are presented as mean±S.E. for three to six independent experiments. Note that values are nanomolar (Table 2 values are micromolar). ^{a}p<0.05 vs. WT DAT for that assay. ^{b}p<0.05 for WIN inhibition vs. dopamine uptake at the same DAT construct. (Both statistical analyses via Student's T test.)

Interestingly, specific binding of [³H]—WIN 35,428 at D79E DAT was undetectable using either suspended cells (the monolayer was scraped from the plate) or a cell membrane preparation in the KRH uptake buffer, whereas [³H]—WIN 35,428 binding affinity at WT DAT was unaffected by these conditions (K_{d}=20±1, 19±1 and 18±4 nM for attached, suspended and membrane-prepared WT DAT CHO cells, respectively). No appreciable specific uptake of [³H]-dopamine was observed for suspended cells possessing either transporter protein (data not shown).

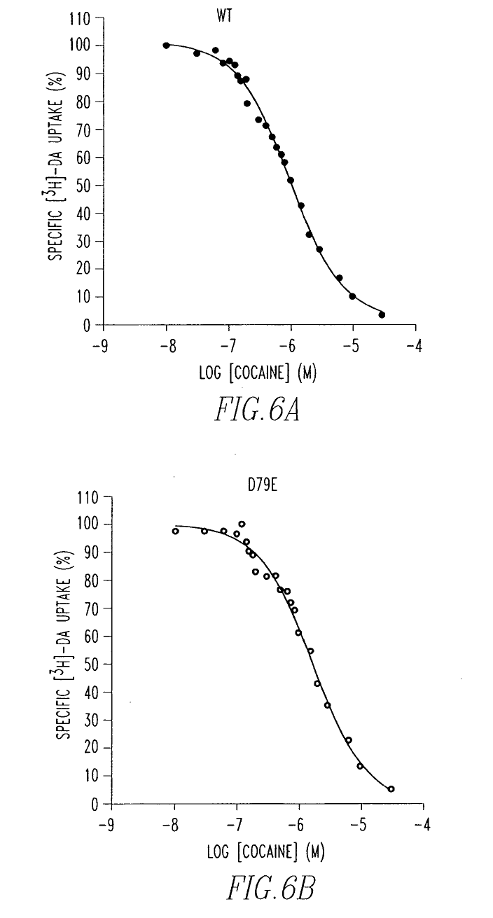

The results with WT DAT cells suggested that a high affinity binding site for uptake inhibitors may have been masked in the dopamine uptake assay. To search for two inhibitor sites at WT DAT, 24-point concentration curves were employed for cocaine inhibition of [³H]-dopamine uptake. A reproducible second WT DAT site was indeed suggested from visual inspection of the curve (but was not sufficiently defined for GraphPad analysis), and this site appeared to be in the vicinity of the 128 nM Ki value previously measured for high affinity binding. To a lesser extent, D79E DAT also occasionally displayed this high affinity cocaine site (FIG. 6). Similarly, DHBA typically displayed a mild deviation from the monophasic uptake inhibition curve for WT DAT that was consistent with its measured higher affinity (800 μM) binding site (data not shown).

In the course of the above work, a surprising implication was that the WT DAT site, conformation or population responsible for higher affinity binding of [³H]—WIN 35,428 was not principally responsible for the observed inhibition of dopamine uptake.

The D79E mutation had no effect on the dopamine uptake inhibition potency of WIN 35,428, mazindol or methylphenidate, and cocaine potency was decreased by only half. The binding affinity for each inhibitor was assessed under conditions identical to those employed in the dopamine uptake inhibition assay, and binding affinity constants matched those for uptake inhibition for D79E DAT. Curiously, this correlation was not seen for WT DAT, as binding affinities were higher than dopamine uptake inhibition potencies for all inhibitors tested (FIG. 5 and Table 3). Moreover, D79E DAT did not display significant [³H]—WIN 35,428 affinity unless cells bearing this protein were attached to the culture plate as a monolayer. The requirement for an attached cell monolayer for [³H]—WIN 35,428 binding at D79E DAT is currently not understood. It is possible that the “off rate” of WIN 35,428 binding at this mutant protein is too rapid for detection with glass-fiber vacuum filtration. Another possibility is that scraping the CHO cells disrupts a DAT association with either another protein, e.g., phosphatase 2A, syntaxin-1A, PICK1 or α-synuclein (Bauman A L et al. “Cocaine and antidepressant-sensitive biogenic amine transporters exist in regulated complexes with protein phosphatase 2A.” J. Neurosci. 20:7571-7578 (2000); Deken S L et al. “Transport rates of GABA transporters: Regulation by the N-terminal domain and syntaxin 1A.” Nat. Neurosci. 3:998-1003 (2000); Torres G E et al. “Functional interaction between monoamine plasma membrane transporters and the synaptic PDZ domain-containing protein PICK1.”Neuron 30:121-134 (2001); Lee F J et al. “Direct binding and functional coupling of alpha-synuclein to the dopamine transporter accelerates dopamine-induced apoptosis.” FASEB J. 15:916-926 (2001)) or itself, as an oligomer (Hastrup H et al. “Symmetrical dimer of the human dopamine transporter revealed by cross-linking Cys-306 at the extracellular end of the sixth transmembrane segment.” Proc. Natl. Acad. Sci. USA 98:10055-10060 (2001); Kilic F et al. “Oligomerization of serotonin transporter and its functional consequences.” Proc. Natl. Acad. Sci. USA 97:3106-3111(2000)), and that such an association is required for 20 nM [³H]—WIN 35,428 binding.

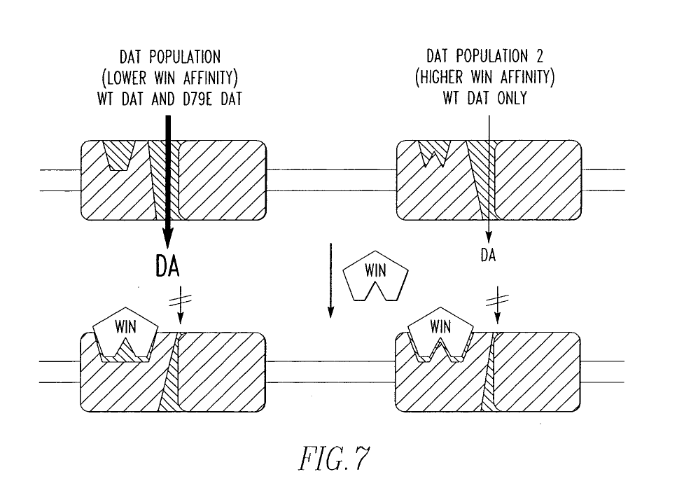

These results suggest that for each uptake inhibitor tested there exists a discrete site, conformation or population for WT DAT that exhibits “higher” or “lower” binding affinity for the drug, and that the D79E mutation largely eliminates the high affinity component. FIG. 7 is a diagram illustrating this scenario, in which two dopamine transporter (DAT) populations (rectangular structures) are represented at the cell surface, within the plasma membrane (parallel horizontal lines). The large majority of [³H]-dopamine uptake is proposed to be mediated by DAT Population 1; Population 2 is thus underrepresented with respect to total uptake of the dopamine radiotracer. Population 1 displays a lower affinity (˜70 nM) for [³H]—WIN 35,428 (“WIN”, inscribed in quasi-pentagonal structure) than does Population 2 (˜20 nM); Population 1 is thus underrepresented with respect to total binding of the WIN 35,428 radiotracer. Higher levels (e.g., >500 nM) of WIN 35,428 (or other DAT inhibitors employed in the study) thoroughly eliminate dopamine uptake by both DAT populations, but the uptake inhibition curves reflect actions principally at Population 1 (the primary [³H]-dopamine conduit). The D79E mutation eliminates DAT Population 2, eliminating the higher affinity binding of DAT inhibitors but affording little or no effect on their dopamine uptake inhibition potencies. Considering that the uptake inhibition potencies for three of four inhibitors 1) were insensitive to the mutation, and 2) matched the binding affinities at D79E DAT, it appears that the lower affinity component for each drug at WT DAT was principally responsible for blocking translocation of dopamine into the cell. It is understandable why the [³H]—WIN 35,428 binding curve for WT DAT did not display 2 obvious phases. In binding displacement assays, use of a low concentration of radioligand favors detection of the site with highest affinity, at the expense of lower affinity sites. Thus, the apparently monophasic WT DAT curve corresponding to the 20 nM K_{d} value may be a weighted average between the lower affinity component and a component with an affinity greater than 20 nM.

On the other hand, such explanations do not account for the disparity between uptake inhibition potency and binding affinity observed for all inhibitors tested at WT DAT. Because the gap between these two values was greatest for cocaine, this drug was chosen for 24-point uptake inhibition experiments that sought to detect a high affinity component. A second phase of the WT DAT curve corresponding to the high affinity value was frequently present using this assay, and occasionally present, and to a lesser extent, in the D79E DAT curve (FIG. 6). While visually apparent, this phase was not sufficiently prominent to be detected with the regression analysis. The WT DAT population that recognizes inhibitors with higher affinity may be a poorer transporter of dopamine than the population containing the lower affinity inhibitor binding site (FIG. 7). This explanation accounts for the underrepresentation of the “high affinity” DAT population in the [³H]-dopamine uptake inhibition assay but not in the [³H]—WIN 35,428 binding assay. Interestingly, syntaxin-1A modulates GABA uptake via direct contact with the GABA transporter (Deken S L et al. “Transport rates of GABA transporters: Regulation by the N-terminal domain and syntaxin 1A.” Nat. Neurosci. 3:998-1003(2000)); it is possible that association of DAT with a cytoskeletal protein (or another DAT molecule) modulates uptake and at the same time creates the higher affinity binding site for classical inhibitors. Alternatively, it cannot be ruled out that the “high affinity” DAT population is a minority for WT DAT and nearly undetectable in the case of D79E DAT.

The lack of correlation between cocaine potencies for binding and uptake inhibition at WT DAT has been previously noted. Using COS cells transiently-transfected with DAT, Pristupa et al. (Pristupa Z B et al. “Pharmacological heterogeneity of the cloned and native human dopamine transporter: Dissociation of [³H]WIN 35,428 and [³H]GBR 12,935 binding. Mol. Pharmacol. 45:125-135 (1994)) reported Kᵢ values of 240 nM and 743 nM for cocaine binding and uptake inhibition, respectively, numbers very close to our own. Using DAT-transfected HEK293 cells, Eshleman et al. (Eshleman A J et al. “Characteristics of drug interactions with recombinant biogenic amine transporters expressed in the same cell type.” J. Pharmacol. Exp. Ther. 289:877-885 (1999)) observed a 2- to 3-fold greater cocaine potency for uptake inhibition than for binding. Literature values for dopamine uptake inhibition potency of cocaine range from 149 nM (Eshleman A J et al. “Characteristics of drug interactions with recombinant biogenic amine transporters expressed in the same cell type” J. Pharmacol. Exp. Ther. 289:877-885 (1999)) to 137,000 nM (Reith MEA et al. “Structure-activity relationships for cocaine congeners in inhibiting dopamine uptake into rat brain synaptic vesicles and bovine chromaffin granule ghosts.” J. Pharmacol. Exp. Ther. 271:1444-1452 (1994)), and this variance appears to be dependent on cell/tissue type and experimental conditions. Again, DAT interactions with DAT binding proteins or DAT posttranslational modifications are among possibilities that may be relevant to the differences in cocaine potency. The inventors have observed cocaine dopamine uptake inhibition potency to fluctuate within a 3-fold range as a function of cell state when employing CHO cells stably transfected with WT DAT.

Despite decades of investigation, the search for a therapeutic against dependence on cocaine has been largely unsuccessful. The principal focus in the development of potential anti-cocaine medications has been to inhibit high affinity cocaine or WIN 35,428 binding while at the same time attempting to spare dopamine uptake. The finding that the potency of high affinity inhibitor binding to WT DAT diverges from that for inhibition of dopamine uptake suggests that development of a “cocaine antagonist” therapeutic should not be focused on blocking high affinity cocaine binding. The D79E DAT mutant can thus serve as an important diagnostic tool in the development of a clinically useful cocaine antagonist.

Whereas particular embodiments of this invention have been described above for purposes of illustration, it will be evident to those skilled in the art that numerous variations of the details of the present invention may be made without departing from the invention as defined in the appended claims.

1. A method of detecting a first compound that interferes with a second compound's ability to inhibit dopamine uptake by a dopamine transporter protein, the first compound not substantially inhibiting dopamine uptake, the method comprising:
providing one or more cells transfected with a mutant dopamine transporter protein and expressing said protein;
contacting said one or more cells with said first and second compounds;
measuring the ability of said first compound to reduce binding of said second compound to said mutant dopamine transporter protein; and
measuring dopamine uptake of said cells in the presence of said first and second compounds.

2. The method of claim 1, wherein said mutant dopamine transporter protein is D79E DAT.

3. The method of claim 1, wherein said one or more cells are CHO cells.

4. The method of claim 1, wherein said second compound is selected from the group consisting of cocaine, cocaine analogs, mazindol and methylphenidate.

5. The method of claim 1, wherein said second compound is cocaine or a cocaine analog.

6. The method of claim 1, wherein said one or more cells are contacted with about 1 picomole to 1 millimole of said first compound, and about 1 nanomole to 1 picomole of said second compound.

7. The method of claim 1, further comprising the steps of:
providing one or more cells transfected with a wild type dopamine transporter protein and expressing said protein;
contacting said one or more cells with said first and second compounds;
measuring the ability of said first compound to reduce binding of said second compound to said wild type dopamine transporter protein; and measuring dopamine uptake of said cells in the presence of said first and second compounds.

8. A method of detecting a first compound that interferes with a second compound's ability to inhibit dopamine uptake by a dopamine transporter protein, the first compound not substantially inhibiting dopamine uptake, the method comprising:
providing one or more cells transfected with a mutant dopamine transporter protein and expressing said protein;
contacting said one or more cells with said first and second compounds; and
measuring the ability of said first compound to reduce binding of said second compound to said mutant dopamine transporter protein

9. The method of claim 8, wherein said mutant dopamine transporter protein is D79E DAT.

10. The method of claim 8, wherein said one or more cells are CHO cells.

11. The method of claim 8, wherein said second compound is selected from the group consisting of cocaine, cocaine analogs, mazindol, and methylphenidate.

12. The method of claim 8, wherein said second compound is cocaine or a cocaine analog.

13. The method of claim 8, wherein said one or more cells are contacted with about 1 picomole to 1 millimole of said first compound, and about 1 nanomole to 1 picomole of said second compound.

14. The method of claim 8, further comprising the steps of
providing one or more cells transfected with a wild type dopamine transporter protein and expressing said protein;
contacting said one or more cells with said first and second compounds; and
measuring the ability of said first compound to reduce binding of said second compound to said wild type dopamine transporter protein.

15. A method of detecting a first compound that interferes with a second compound's ability to inhibit dopamine uptake by a dopamine transporter protein, the first compound not substantially inhibiting dopamine uptake, the method comprising:
providing one or more cells transfected with a mutant dopamine transporter protein and expressing said protein;
contacting said one or more cells with said first and second compounds; and
measuring dopamine uptake of said cells in the presence of said first and second compounds.

16. The method of claim 15, wherein said mutant dopamine transporter protein is D79E DAT.

17. The method of claim 15, wherein said one or more cells are CHO cells.

18. The method of claim 15, wherein said second compound is selected from the group consisting of cocaine, cocaine analogs, mazindol, and methylphenidate.

19. The method of claim 15, wherein said second compound is cocaine, or a cocaine analog.

20. The method of claim 15, wherein said one or more cells are contacted with about 1 picomole to 1 millimole of said first compound, and about 1 nanomole to 1 picomole of said second compound.

21. The method of claim 15, further comprising the steps of:
providing one or more cells transfected with a wild type dopamine transporter protein and expressing said protein;
contacting said one or more cells with said first and second compounds; and
measuring dopamine uptake of said cells in the presence of said first and second compounds.
